(19) United States
(12) Patent Application Publication
(10) Pub. No.: US 2004/0110757 A1
(43) Pub. Date: Jun. 10, 2004

(54) Flt-1 ligands and their uses in the treatment of diseases regulatable by angiogenesis

(76) Inventors: Thomas Arrhenius, Del Mar, CA (US); Yujin Huang, San Diego, CA (US); Lin Zhang, San Diego, CA (US); Rossy Serafimov, Metz (FR); Alex Nadzan, Encinitas, CA (US); Dominic G Spinella, Santee, CA (US)

Correspondence Address: Daniel W Collins, Chugai Pharma USA, 6275 Nancy Ridge Drive, San Diego, CA, 92121, US

(21) Appl. No.: 10/472,631

(22) PCT Filed: Mar. 21, 2002
(86) PCT No.: PCT/US02/08862

(51) Int. Cl. A61K 31/53; A61K 31/506; A61K 31/4439; A61K 31/4245; A61K 31/4184

(52) U.S. Cl. 514/241; 514/256; 514/338; 514/339; 514/364; 514/394; 514/416

(57) ABSTRACT

This invention is a method of treatment and prophylaxis of diseases regulatable by angiogenesis, including novel FLT-1 ligands and pharmaceutical compositions containing them, useful in the methods for the treatment and prophylaxis of these diseases, as well as intermediates and processes useful for the preparation of the compounds of the invention as claimed.

### FIELD OF THE INVENTION

The present invention relates to methods of treating diseases regulatable by angiogenesis, and to novel compounds and their prodrugs, and/or pharmaceutically acceptable salts, pharmaceutical compositions containing such compounds useful in treating such diseases.

### BACKGROUND

Vascular endothelial cell growth factor (VEGF) is an endothelial cell mitogen and permeability factor that serves as a primary mediator of angiogenesis, or the sprouting of new blood vessels from existing vasculature in the adult organism (Ferrara N, Davis-Smyth T. Endocr. Rev. 18:4-25, 1997). In addition, VEGF plays a critical role in vasculogenesis, or the development of the vascular system in utero. Both angiogenesis and vasculogenesis are profoundly inhibited in experimental conditions in which VEGF levels are reduced or VEGF activity is neutralized. The cellular functions of VEGF are mediated by its binding to two receptors, Flt-1 or VEGFR1 and Flk-1/KDR or VEGFR2, both of which are expressed on the surface of endothelial cells. Flt-1 and KDR are members of the type III receptor tyrosine kinase family of proteins with seven extracellular IgG-like repeats, a single transmembrane domain, and an intracellular tyrosine kinase domain (Mustonen T, Alitalo K. J. Cell Biol. 129:895-898, 1995). The binding affinity of VEGF to Flt-1 is approximately ten-fold higher than its binding to KDR. There is evidence that binding of VEGF to Flt-1 may play an important role in VEGF-mediated angiogenesis and vasculogenesis. Ribozymes targeting Flt-1 mRNA inhibit VEGF-induced human endothelial cell proliferation and demonstrate anti-angiogenic activity in the corneal pocket model of VEGF-induced angiogenesis in vivo (Parry T J, Cushman C, Gallegos A M, Agrawal A B, Richardson M, Andrews L E, Maloney L, Mokler V R, Wincott F E, Pavco P A: Nucleic Acids Research 27:2569-2577, 1999). Neutralizing monoclonal antibodies directed against Flt-1 inhibit actin reorganization and migration of endothelial cells in culture (Kanno S, Oda N, Abe M, Terai Y, Ito M, Shitara K, Tabayashi K, Shibuya M, Sato, Y. Oncogene 19:2138-2146, 2000). In addition, mice homozygous for a targeted Flt-1 mutation exhibit abnormal embryonic vascular channels, with death of embryos at E8.5 resulting from a failure of vasculogenesis (Fong G H, Rossant J, Gertsenstein M, Breitman M L. Nature 376:66-70, 1995). Thus, while there is a large body of evidence supporting a critical role for KDR in angiogenesis and vasculogenesis, these studies suggest that Flt-1 also mediates these VEGF-dependent processes.

A number of pathological conditions are associated with ongoing angiogenesis in the lesion. The growth of tumors beyond 1-2 mm in diameter is dependent on the induction of angiogenesis within the tumor to allow adequate supply of nutrients and oxygen and removal of metabolic waste products (Folkman J, in The Molecular Basis of Cancer, pp 206-232, 1995). There is a large body of evidence that anti-angiogenic agents inhibit the growth of primary and metastatic tumors in a variety of animal models. Ribozymes targeting Flt-1 significantly inhibit the growth of Lewis Lung tumors at the primary site as well as the formation of lung metastases (Pavco P A, Bouhana K S, Gallegos. A M, Agrawal A, Blanchard K S, Grimm S L, Jensen K L, Andrews L E, Wincott F E, Pitot P A, Tressler R J, Cushman C, Reynolds M A, Parry T J. Clinical Cancer Res. 6:2094-2103, 2000). In the KM12L4a human colon cancer model, anti-Flt-1 ribozyme inhibits liver metastasis formation after intrasplenic inoculation (loc. cit.). Diabetic retinopathy and macular degeneration are ocular diseases in which impaired vision is associated with intraocular vascular proliferation. In rheumatoid arthritis, the synovium is characterized by the formation of a highly vascularized pannus that degrades articular cartilage; levels of VEGF are elevated in the synovial fluid of patients with this disease. In the skin disease psoriasis, characterized by hyperplasia and abnormal differentiation of epidermal keratinocytes, there is a markedly increased vascularity in the dermis and VEGF expression is elevated. In all of these pathological conditions associated with angiogenesis, inhibition of this process with the use of inhibitors of VEGF binding to its receptor Flt-1 may have therapeutic benefit.

The compounds described in this patent may act functionally as antagonists or agonists of the Flt-1 receptor. If they serve as Flt-1 agonists, compounds described in this invention may stimulate angiogenesis and therefore have utility in the treatment of diseases and conditions in which stimulation of angiogenesis may be beneficial, such as wound healing and ulcers.

### SUMMARY OF THE INVENTION

The present invention comprises a method of treatment and prophylaxis of diseases related to angiogenesis. Accordingly, the invention also comprises novel pharmaceutical compositions comprising the compounds, useful in the methods for the prophylaxis and treatment of diseases, described above. Novel compounds and compositions of the invention and intermediates and processes useful for the preparation of the compounds of the invention are also disclosed and claimed as part of this invention.

The novel compounds of the invention are represented by the following general structure:

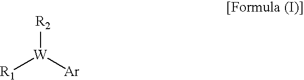

Wherein

W is an aromatic core selected from phenyl, pyridyl, pyrimidyl, triazyl, and oxadiazolyl

Ar is an aromatic substituent, which may be substituted or unsubstituted. Where Ar is substituted, it preferably has up to three substituents, and preferably such substituents are in the meta or para position relative to the attachment of Ar to the core molecule. Preferred substituents are amino, alkylamino, dialkylamino, lower alkoxy, nitro, carboxy, hydroxy, alkoxy alkyl, alkoxy alkoxy, alkylthio, haloalkyl, more preferably fluoroalkyl, halo, preferably, bromo, chloro, and fluoro, lower alkyl, phenyl, pyridyl, an ester, or an amide. More preferred are polar or hydrophilic substituents, including methoxy, nitro, carboxy, hydroxy, amino, alkylamino, and the like.

R₁ is selected from,

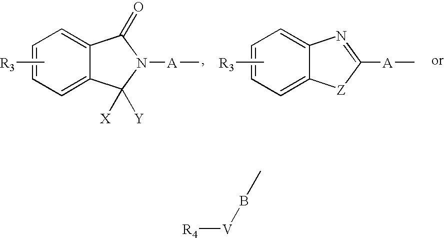

Wherein A is a bond or a spacer selected from phenyl, substituted phenyl wherein substitution is selected from methoxy, or other small substituents, preferably unsubstituted phenyl. amino, amido, ester, oxy, and the like. Preferably when phenyl is the spacer, the phenyl spacer is attached to the molecule in the meta or para position.

B is a linker selected from oxy, alkoxy, aryl carbonyl, arylcarbonylamino, a bond, amido, carbonyloxy, oxycarbonyl.

V is an aryl group, preferred V include phenyl, furyl, thienyl, pyridyl, and pyrrolyl. Preferably V is phenyl. Preferred substitution on V is in the 2- or 4- position relative to the point of attachment to the B moiety.

X and Y are hydrogen, or together form oxo, preferably X and Y are oxo.

Z is selected from oxygen, nitrogen, and sulfur.

R₂ is selected from Ar, hydrogen, hydroxy, halo, carboxy or R₁

R₃ is selected from one or two of hydroxy, alkoxy, nitro, sulfoxy, carboxy ester, or an amide or such radicals connected to the substituent via a lower alkyl. In the absence of R₃ the ring is substituted only by hydrogen. Preferably only one R₃ appears, and more preferably such R₃ is carboxy.

R₄ is one or two of amino, alkylamino, dialkylamino, loweralkoxy, nitro, carboxy, hydroxy, alkoxy alkyl, alkoxy alkoxy, alkylthio, haloalkyl, more preferably fluoroalkyl, halo, preferably, bromo, chloro, and fluoro, lower alkyl, phenyl, pyridyl, an ester, or an amide. More preferred are polar or hydrophilic substituents, including methoxy, nitro, carboxy, hydroxy, amino, alkylamino, and the like. R₄ can also be represented as —OCH₂O—. Where one R₄ appears, the position where another R₄ may have appeared is substituted by hydrogen.

Wherein at least one of R₃ and R₄ must be carboxy, or a carboxy substituted radical.

Preferably R₂, Ar and R₁ are attached to W via carbons which are not on adjacent atoms in the ring.

The compositions of the invention comprise:

(a) a safe and effective amount of an angiogenesis inhibiting compound, prodrug thereof or pharmaceutical salt thereof; and

(b) a pharmaceutically-acceptable carrier.

As discussed above, numerous diseases can be mediated by such related therapy. Thus, the compounds of the invention are useful in therapy with regard to conditions involving this antiangiogenic activity.

The invention's compounds can therefore be formulated into pharmaceutical compositions for use in treatment or prophylaxis of these conditions. Standard pharmaceutical formulation techniques are used, such as those disclosed in Remington's Pharmaceutical Sciences, Mack Publishing Company, Easton, Pa., latest edition.

A “safe and effective amount” of a compound of the invention is an amount that is effective, to alter the process of angiogenisis at the site(s) of activity, in a subject, a tissue, or a cell, and preferably in an animal, more preferably in a mammal, without undue adverse side effects (such as toxicity, irritation, or allergic response), commensurate with a reasonable benefit/risk ratio, when used in the manner of this invention. The specific “safe and effective amount” will, obviously, vary with such factors as the particular condition being treated, the physical condition of the patient, the duration of treatment, the nature of concurrent therapy (if any), the specific dosage form to be used, the carrier employed, the solubility of the compound therein, and the dosage regimen desired for the composition.

In addition to the subject compound, the compositions of the subject invention contain a pharmaceutically-acceptable carrier. The term “pharmaceutically-acceptable carrier”, as used herein, means one or more compatible solid or liquid filler diluents or encapsulating substances which are suitable for administration to a mammal. The term “compatible”, as used herein, means that the components of the composition are capable of being commingled with the subject compound, and with each other, in a manner such that there is no interaction which would substantially reduce the pharmaceutical efficacy of the composition under ordinary use situations. Pharmaceutically-acceptable carriers must, of course, be of sufficiently high purity and sufficiently low toxicity to render them suitable for administration preferably to an animal, preferably mammal being treated.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

If the subject compound is to be injected, the preferred pharmaceutically-acceptable carrier is sterile, physiological saline, with blood-compatible suspending agent, the pH of which has been adjusted to about 7.4. In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically-acceptable carrier, in compositions for parenteral administration, comprises at least about 90% by weight of the total composition.

The compositions of this invention are preferably provided in unit dosage form. As used herein, a “unit dosage form” is a composition of this invention containing an amount of a compound that is suitable for administration to an animal, preferably mammal subject, in a single dose, according to good medical practice. (The preparation of a single or unit dosage form however, does not imply that the dosage form is administered once per day or once per course of therapy. Such dosage forms are contemplated to be administered once, twice, thrice or more per day, and are expected to be given more than once during a course of therapy, though a single administration is not specifically excluded. The skilled artisan will recognize that the formulation does not specifically contemplate the entire course of therapy and such decisions are left for those skilled in the art of treatment rather than formulation.) These compositions preferably contain from about 5 mg (milligrams), more from about 10 mg to about 1000 mg, more preferably to about 500 mg, most preferably from to about 300 mg, of the selected compound.

The compositions of this invention may be in any of a variety of forms, suitable (for example) for oral, nasal, rectal, topical (including transdermal), ocular, intracereberally, intravenous, intramuscular, or parenteral administration. (The skilled artisan will appreciate that oral and nasal compositions comprise compositions that are administered by inhalation, and made using available methodologies.) Depending upon the particular route of administration desired, a variety of pharmaceutically-acceptable carriers well-known in the art may be used. These include solid or liquid fillers, diluents, hydrotropes, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods of this invention are described in the following references, all incorporated by reference herein: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976).

Various oral dosage forms can be used, including such solid forms as tablets, capsules, granules and bulk powders. These oral forms comprise a safe and effective amount, usually at least about 5%, and preferably from about 25% to about 50%, of the compound. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of the subject invention, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions of the subject invention may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions preferably comprise a safe and effective amount, usually at least about 0.1%, and preferably from about 1% to about 5%, of the compound. Suitable carriers for topical administration preferably remain in place on the skin as a continuous film, and resist being removed by perspiration or immersion in water. Generally, the carrier is organic in nature and capable of having dispersed or dissolved therein the compound. The carrier may include pharmaceutically-acceptable emolients, emulsifiers, thickening agents, solvents and the like.

Methods of Administration

The compounds and compositions of this invention can be administered topically or systemically. Systemic application includes any method of introducing compound into the tissues of the body, e.g., intra-articular, intrathecal, epidural, intramuscular, transdermal, intravenous, intraperitoneal, subcutaneous, sublingual administration, or by inhalation, rectal, or oral administration. The compounds of the present invention are preferably administered orally.

The specific dosage of the compound to be administered, as well as the duration of treatment, and whether the treatment is topical or systemic are interdependent. The dosage and treatment regimen will also depend upon such factors as the specific compound used, the treatment indication, the ability of the compound to reach minimum concentrations at the site of Flt-1, the personal attributes of the subject (such as weight), compliance with the treatment regimen, and the presence and severity of any side effects of the treatment.

Typically, for a human adult (weighing approximately 70 kilograms), from about 5 mg, preferably from about 10 mg to about 3000 mg, more preferably to about 1000 mg, more preferably to about 100 mg, of compound are administered per day for systemic administration. It is understood that these dosage ranges are by way of example only, and that daily administration can be adjusted depending on the factors listed above.

A preferred method of administration for treatment is orally, by inhalation, or parenterally. As is known and practiced in the art, all formulations for parenteral administration must be sterile. For mammals, especially humans, (assuming an approximate body weight of 70 kilograms) individual doses of from about 10 mg to about 1000 mg are preferred.

A preferred method of systemic administration is oral. Individual doses of from about 10 mg to about 1000 mg, preferably to about 300 mg are preferred.

Topical administration can be used to deliver the compound systemically, or to treat a subject locally. The amounts of compound to be topically administered depends upon such factors as skin sensitivity, type and location of the tissue to be treated, the composition and carrier (if any) to be administered, the particular compound to be administered, as well as the particular disorder to be treated and the extent to which systemic (as distinguished from local) effects are desired.

For localized conditions, topical administration is preferred. When so formulated, the compounds of the invention can be formulated with or without other actives as gels, drops or ointments, or can be incorporated into collagen or a hydrophilic polymer shield. The materials can also be inserted as a contact lens or reservoir or as a subconjunctival formulation. For treatment of skin, the compound is applied locally and topically, in a gel, paste, salve or ointment. The mode of treatment thus reflects the nature of the condition and suitable formulations for any selected route are available in the art.

In all of the foregoing, of course, the compounds of the invention can be administered alone or as mixtures, and the compositions may further include additional drugs or excipients as appropriate for the indication. For example, in the treatment of cardiovascular diseases, it is clearly contemplated that the invention may be used in conjunction with beta-blockers, calcium antagonists, ACE inhibitors, or known cardiovascular drugs or therapies. Hence, in this example, novel compounds or compositions of this invention are useful when dosed together with another active and can be combined in a single dosage form or composition.

### Definitions

Examples of alkyl include

As used herein, “alkyl” means a cyclic, branched, or straight chain alkanyl or alkenyl substituent containing only carbon and hydrogen, such as pentyl, octyl, cyclohexylethyl, heptyl, as well as butyl, propyl, isopropyl, ethyl, methyl. pentyl, and adamantyl. Alkyl groups can be saturated or unsaturated (e.g., containing —C═C— or —C═C— linkages), at one or several positions. Typically, alkyl groups will comprise 1 to 12 carbon atoms, preferably 1 to 10, and more preferably 1 to 8 carbon atoms.

As used herein, “lower alkyl” means subset of alkyl, and thus is a hydrocarbon substituent, which is linear or branched. Preferred lower alkyls are of 1 to about 6 carbons, and may be branched or linear, and may include cyclic substituents, either as part or all of their structure. Examples of lower alkyl include butyl, propyl, isopropyl, ethyl, and methyl. Likewise this term may be combined with other art accepted terms. For example “lower alkoxy” means alkoxy as understood in the art, wherein the alkyl portion of the substituent is lower alkyl.

As used herein, “aryl” means a substituted or unsubstituted aromatic radical having a single-ring (e.g., phenyl) or multiple condensed rings (e.g., naphthyl or anthryl), which can be optionally unsubstituted or substituted with amino, cyano, hydroxyl, lower alkyl, haloalkyl, alkoxy, nitro, halo, mercapto, and other substituents, and which may or may not include one or more heteroatoms. Hence the term “heteroaryl” is clearly contemplated in the term “aryl”. Preferred carbocyclic aryl, is phenyl. Preferred monocyclic heterocycles are 5 or 6 membered rings. Preferably where the term aryl represents a heterocycle, it is referred to as “heteroaryl”, and has one or more heteroatom(s). Preferred numbers of such heteroatoms are from one to three N atoms, and preferably wherein when “heteroaryl” is a heterocycle of five members, it has one or two heteroatoms selected from O, N, or S. Hence, preferred heterocycles have up to three, more preferably two or less, heteroatoms present in the aromatic ring. The skilled artisan will recognize that among heteroaryl, there are both five and six membered rings. Examples of “heteroaryl” include; thienyl, pyridyl, pyrimidyl, pyridazyl, furyl, oxazolyl, imidazolyl, thiazolyl, oxadiazilyl, triazinyl, triazolyl, thiadiazolyl, and others, which the skilled artisan will recognize. In this definition it is clearly contemplated that substitution on the aryl ring is within the scope of this invention. Where substitution occurs, the radical is called a substituted aryl. Though many substituents will be useful, preferred substituents include those commonly found in aryl compounds, such as alkyl, hydroxy, alkoxy, cyano, nitro, halo, haloalkyl, mercapto and the like. Such substituents are prepared using known methodologies.

As used herein, the term “amide” includes both RNR′CO— (in the case of R=alkyl, alkaminocarbonyl-) and RCONR′— (in the case of R=alkyl, alkyl carbonylamino-).

As used herein, the term “ester” includes both ROCO— (in the case of R=alkyl, alkoxycarbonyl-) and RCOO— (in the case of R=alkyl, alkylcarbonyloxy-).

A substituent described as a radical in this specification may form a ring with another radical as described herein. When such radicals are combined, the skilled artisan will understand that there are no unsatisfied valences in such a case, but that specific substitutions, for example a bond for a hydrogen, is made. Hence certain radicals can be described as forming rings together. The skilled artisan will recognize that such rings can and are readily formed by routine chemical reactions, and it is within the purview of the skilled artisan to both envision such rings and the methods of their formations. Preferred are rings having from 3-7 members, more preferably 5 or 6 members. Compounds described herein may have cyclic structures therein, such as a ring formed by the combination of two radicals. In that regard the skilled artisan recognizes that this method of description is routine in medicinal chemistry, though such may not rigorously reflect the chemical synthetic route. As used herein the term “ring” or “rings” when formed by the combination of two radicals refers to heterocyclic or carbocyclic radicals, and such radicals may be saturated, unsaturated, or aromatic. For example, preferred heterocyclic ring systems include heterocyclic rings, such as morpholinyl, piperdinyl, imidazolyl, pyrrolidinyl, and pyridyl.

The skilled artisan will recognize that some structures described herein may be resonance forms or tautomers of compounds that may be fairly represented by other chemical structures, even when kinetically, the artisan recognizes that such structures are only a very small portion of a sample of such compound(s). Such compounds are clearly contemplated within the scope of this invention, though such resonance forms or tautomers are not represented herein.

In addition, prodrugs where a group is removed by a biological process in situ clearly contemplates that compounds of the invention, as well as tautomers trapped as prodrugs can be provided. “Prodrug”, as used herein is any compound wherein when it is exposed to the biological processes in an organism, is hydrolyzed, metabolized, derivatized or the like, to yield an active substance having the desired activity. The skilled artisan will recognize that prodrugs may or may not have any activity as prodrugs. It is the intent that the prodrugs described herein have no deleterious effect on the subject to be treated when dosed in safe and effective amounts. These include for example, biohydrolyzable amides and esters. A “biohydrolyzable amide” is an amide compound which does not essentially interfere with the activity of the compound, or that is readily converted in vivo by a cell, tissue, or human, mammal, or animal subject to yield an active compound of the invention. A “biohydrolyzable ester” refers to an ester compound of the invention that does not interfere with the activity of these compounds or that is readily converted by an animal to yield an active formula (I) compound. Such biohydrolyzable prodrugs are understood by the skilled artisan and are embodied in regulatory guidelines.

Compounds and compositions herein also specifically contemplate pharmaceutically acceptable salts, whether cationic or anionic. A “pharmaceutically-acceptable salt” is an anionic salt formed at any acidic (e.g., carboxyl) group, or a cationic salt formed at any basic (e.g., amino) group. Many such salts are known in the art, as described in World Patent Publication 87/05297, Johnston et al., published Sep. 11, 1987 (incorporated by reference herein). Preferred counter-ions of salts formable at acidic groups can include cations of salts, such as the alkali metal salts (such as sodium and potassium), and alkaline earth metal salts (such as magnesium and calcium) and organic salts. Preferred salts formable at basic sites include anions such as the halides (such as chloride salts). Of course, the skilled artisan is aware that a great number and variation of salts may be used, and examples exist in the literature of either organic or inorganic salts useful in this manner.

Inasmuch as the compounds of the invention may contain optical centers, “Optical isomer”, “stereoisomer”, “enantiomer,” “diastereomer,” as referred to herein have the standard art recognized meanings (cf. Hawleys Condensed Chemical Dictionary, 11th Ed.) and are included in the compounds claimed, whether as racemates, or their optical isomers, stereoisomers, enantiomers, diastereomers.

### Preparation of Compounds of the Invention

The starting materials used in preparing the compounds of the invention are known, made by known methods, or are commercially available as a starting material. It will be apparent to the skilled artisan that methods for preparing precursors and functionality related to the compounds claimed herein are generally described in the literature. The skilled artisan given the literature and this disclosure is well equipped to prepare any of the claimed compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 2^{nd} Ed., John Wiley & Sons (1991).

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure.

The compounds of the invention can be prepared according to the general methods, outlined in Schemes 1-3.

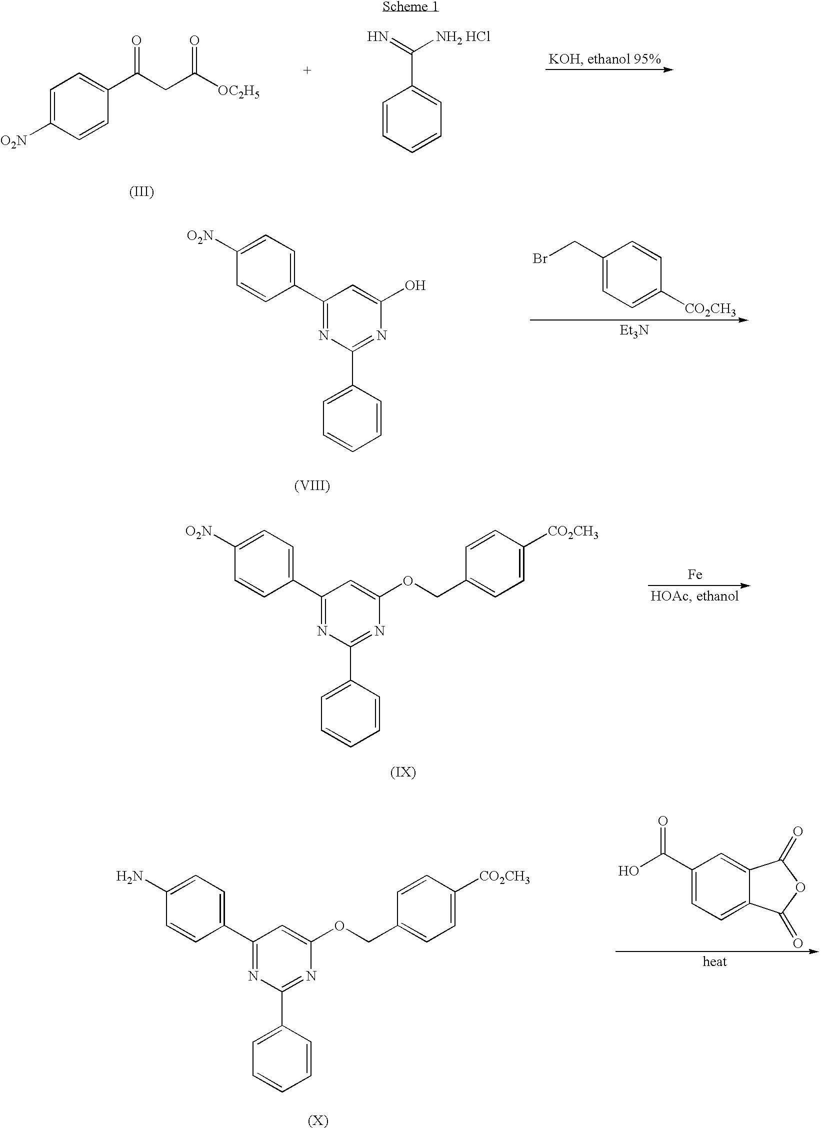

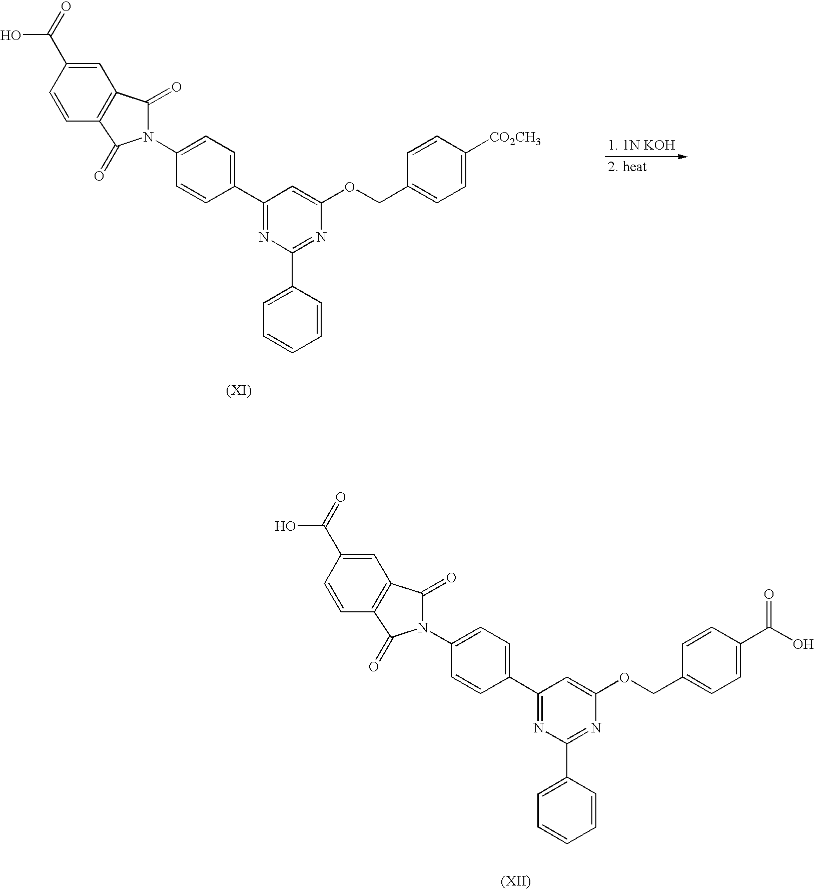

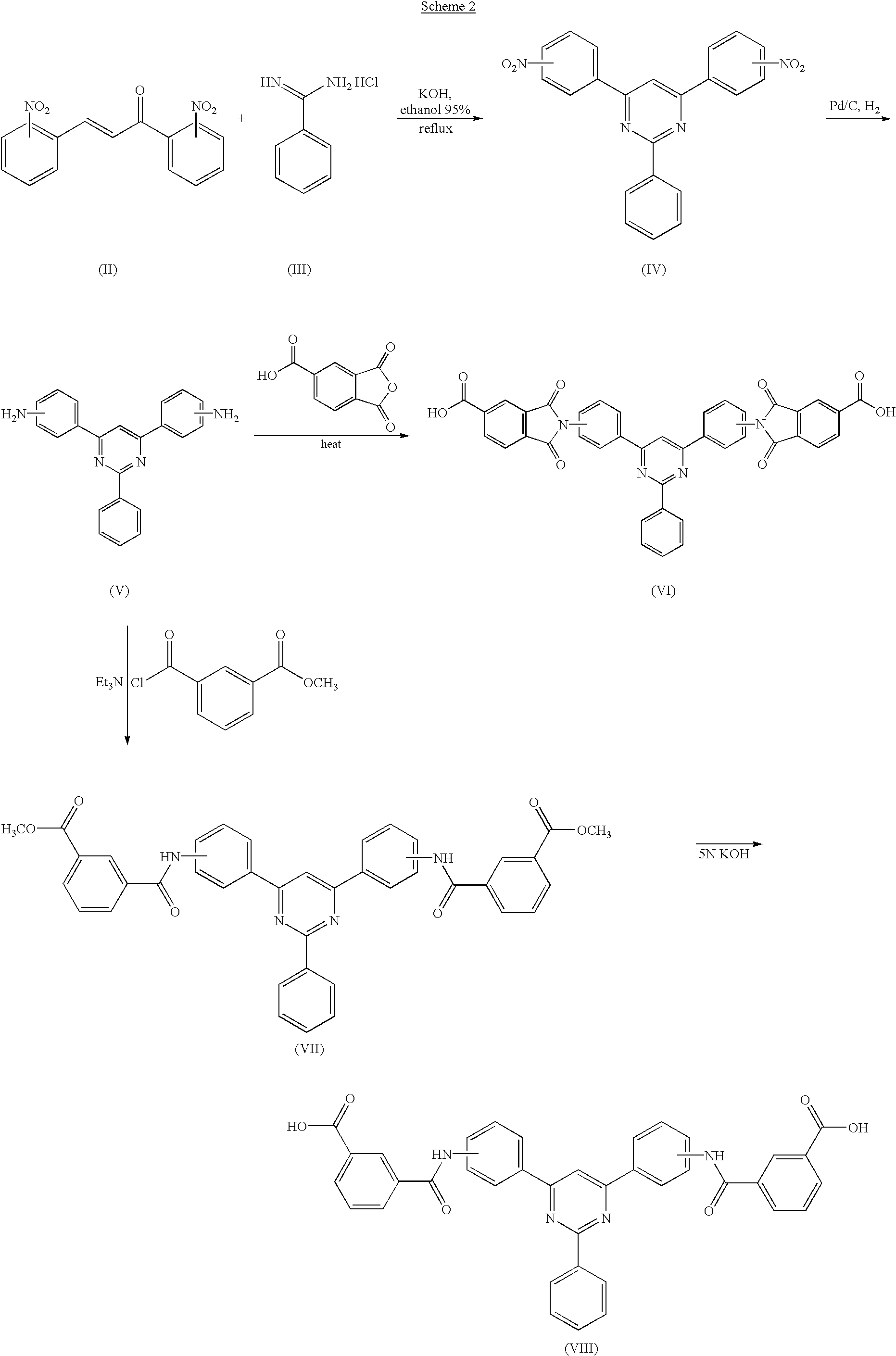

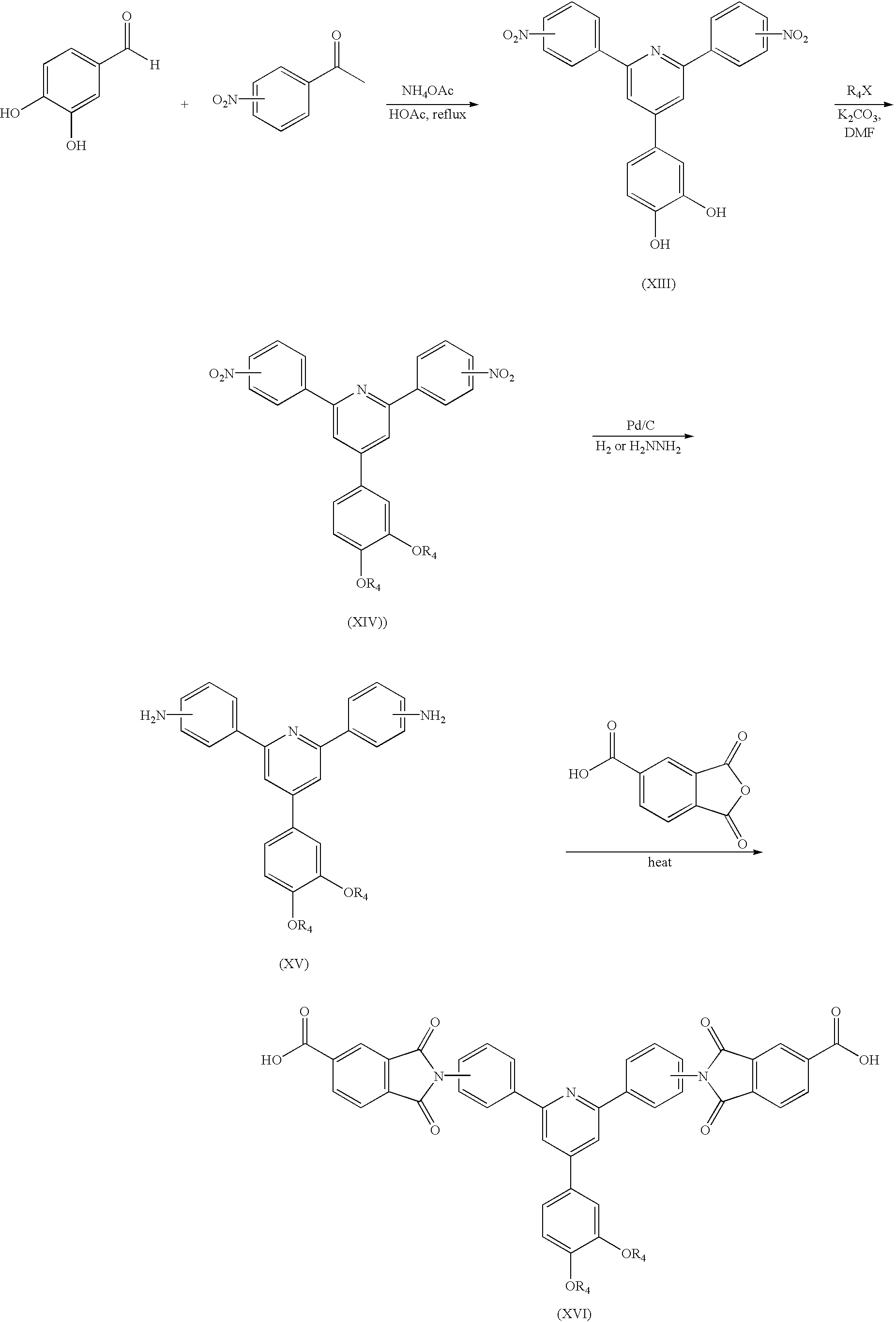

### EXAMPLES

To further illustrate this invention, the following examples are included. The examples should not, of course, be construed as specifically limiting the invention. Variations of these examples within the scope of the claims are within the purview of one skilled in the art are considered to fall within the scope of the invention as described, and claimed herein. The reader will recognize that the skilled artisan, armed with the present disclosure, and skill in the art is able to prepare and use the invention without exhaustive examples.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

¹H nuclear magnetic resonance spectra (NMR) is measured in DMSO-d6 or other solvents as indicated by a Varian NMR spectrometer (Unity Plus 400, 400 MHz for ¹H) unless otherwise indicated and peak positions are expressed in parts per million (ppm) downfield from tetramethylsilane. The peak shapes are denoted as follows, s, singlet; d, doublet; t, triplet; q, quartet; m, multiplet.

The following abbreviations have the indicated meanings:

CH₂Cl₂=dichloromethane

CHCl₃=chloroform

DMAP=4-(dimethylamino)-pyridine

DMF=N,N-dimethylformamide

DMSO=dimethylsulfoxide

ESIMS=electron spray mass spectrometry

EtOAc=ethyl acetate

hrs.=hours

MgSO₄=magnesium sulfate

Pd/C=palladium on activated carbon

r.t.=room temperature

THF=tetrahydrofuran

### Example 1

Preparation of 2-(4-{6-[(4-Carboxybenzyl)Oxy]-2-Phenylpyrimidin-4-Yl}Phenyl)-1,3-Dioxoisoindoline-5-Carboxylic Acid

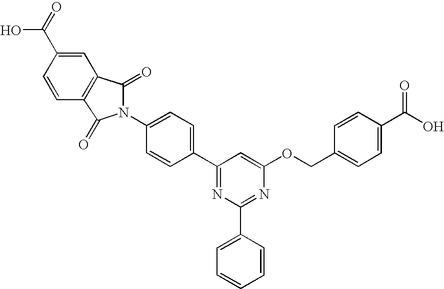

Step 1

Ethyl 4-nitrobenzoylacetate (5.0 g, 21 mmol), benzamidine hydrochloride (3.3 g, 21 mmol), potassium hydroxide (2.75 g, 42 mmol) and 95% ethanol (100 ml) were placed in a 250-ml round-bottomed flask. The suspension was heated to reflux for 5 hours. The reaction mixture was then cooled to room temperature and quenched with water. The mixture was acidified with 1 N HCl and a solid precipitated. The precipitate was washed with water and ethanol and recrystalized from acetic acid to afford 6-(4-nitrophenyl)-2-phenylpyrimidin4-ol (5.4 g, 88% yield).

Step 2

To a solution of 6-(4-nitrophenyl)-2-phenylpyrimidin-4-ol (1.0 g, 3.4 mmol) in DMF (10 ml) was added methy 4-(bromomethyl) benzoate (0.86 g, 3.75 mmol) and triethylamine (0.52 ml, 3.75 mmol). The reaction mixture was heated to 80° C. for 1 hour and then cooled to room temperature. The mixture was diluted with water and acidified with 1N HCl. The precipitate was washed with water and dried to provide Methyl4-({[6-(4-nitrophenyl)-2-phenylpyrimidin4-yl]oxy}methyl)benzoate (1.4 g, 93% yield).

Step 3

Methyl-4-({([6-(4-nitrophenyl)-2-phenylpyrimidin-4-yl]oxy}methyl)benzoate (500 mg, 1.13 mmol), iron (76 mg, 1.36 mmol), acetic acid (408 mg, 6.8 mmol) and ethanol (10 ml) were placed in a 50-ml round-bottomed flask. The suspension was heated to reflux for 8 hours. The reaction mixture was then cooled to room temperature and quenched with water. The precipitate was washed with water and ethanol. The crude product was chromatographed on silica gel to give methyl-4-({[6-(4-aminophenyl)-2-phenylpyrimidin-4-yl]oxy}methyl)benzoate (450 mg, 97% yield).

Step 4

Methyl4-({[6-(4-aminophenyl)-2-phenylpyrimidin-4-yl]oxy}methyl)benzoate (75 mg, 0.18 mmol), 1, 2, 4-benzentricarboxylic anhydride (35 mg, 0.18 mmol) and toluene (5 ml) were placed in a 15-ml round-bottomed flask. The suspension was heated to 150° C. for 30 minutes and cooled to room temperature. The precipitate was washed with CHCl₃ to yield 2-[4-(6-{[4-(methoxycarbonyl)benzyl]oxy}-2-phenylpyrimidin-4-yl)phenyl]-1,3-dioxoisoindoline-5-carboxylic acid (69.4 mg, 66% yield).

Step 5

To a solution of 2-[4-(6-{[4-(methoxycarbonyl)benzyl]oxy}-2-phenylpyrimidin-4-yl)phenyl]-1,3-dioxoisoindoline-5-carboxylic acid (54 mg, 0.092 mmol) in THF (4 ml) was added 1 ml 1N aqueous KOH solution. The mixture solution was stirred at room temperature for 24 hrs and then acidified with 20% aqueous HCl solution. The precipitate was washed with water and heated to 200° C. for 5 minters and cooled down to room temperature. The solid was washed with CHCl₃ to produce the title compound (18.8 mg, 35% yield). ¹H NMR (DMSO-d6) δ: 5.7 (s, 2H), 7.55 (m, 3H), 7.58 (1s, 1H), 7.64 (d, 2H, J=8.0 Hz), 7.65 (d, 2H, J=8.0 Hz), 7.96 (d, 2H, J=7.6 Hz), 8.08 (d, 1H, J=8.4 Hz), 8.31 (s, 1H), 8.35 (d, 1H, J=8.4 Hz), 8.43 (d, 2H, J=8.0 Hz), 8.51 (m, 2H); ESIMS: m/z 572 (M+H).

### Example 2

Preparation of 3-({[4-(6-{4-[(3-Carboxybenzoyl)Amino]Phenyl}-2-Phenylpyrimidin-4-Yl)Phenyl]Amino}Carbonyl)Benzoic Acid

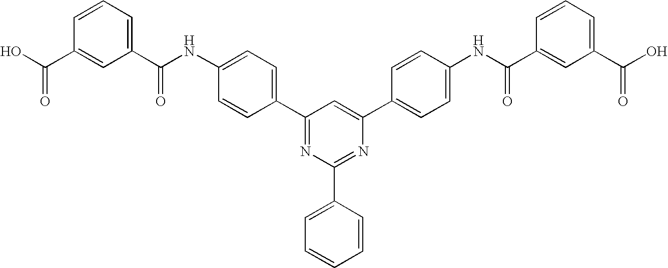

Step 1

To a solution of 4-nitroacetophenone (1.0 g, 6.06 mmol) and 4-nitrobenzaldehyde (0.92 g, 6.06 mmol) in acetic acid (25 ml) was added 5 drops of H₂SO₄ at room temperature. The mixture was heated to reflux for 30 minutes. The reaction mixture was then cooled to room temperature and quenched with water. Solvent was removed under reduced pressure. The mixture was neutralized with 5N NaOH. The precipitate was filtered, washed with water and then recrystallized from EtOAc/hexanes to give (2E)-1,3-bis(4-nitrophenyl)prop-2-en-1-one (1.7 g, 94% yield).

Step 2

To a solution of (2E)-1,3-bis(4-nitrophenyl)prop-2-en-1-one (0.5 g, 1.67 mmol) and benzamidine hydrochloride (0.13 g, 8.3 mmol) in 95% alcohol (10 ml) was added potassium hydroxide (93 mg, 1.67) in 95% alcohol (10 ml). The resulting mixture was heated to reflux on a steam-bath for three hours. A solid precipitated from solution after the reaction mixture was cooled to room temperature. The precipitate was thoroughly washed with warm water to remove the inorganic salts followed by warm alcohol. The solid was recrystallized from glacial acetic acid to afford 4,6-bis(4-nitrophenyl)-2-phenylpyrimidine (0.27 g, 82% yield).

Step 3

To a solution of 4,6-bis(4-nitrophenyl)-2-phenylpyrimidine(0.5 g, 1.25 mmol) in acetic acid (8 ml) was added a catalytic amount of Pd/C under nitrogen. The mixture was bubbled with H₂ gas and stirred for 3 hrs at room temperature. The reaction mixture was filtered through celite. The filtrate was diluted with EtOAc and washed with water and brine, dried over MgSO₄ and concentrated to provide 4-[6-(4-aminophenyl)-2-phenylpyrimidin4-yl]aniline (390 mg, 92% yield).

Step 4

To a solution of 4-[6-(4-aminophenyl)-2-phenylpyrimidin-4-yl]aniline (100 mg, 0.3 mmol) in THF (5 ml) was added methyl 3-(chlorocarbonyl) benzoate (117 mg, 0.59 mmol) and triethylamine (0.091 ml, 0.65 mmol) at room temperature. The reaction mixture was stirred for 30 mintes and quenched with water. The precipitate was filtered and washed with water to give methyl-3-[({4-[6-(4-{[3-(methoxycarbonyl)benzoyl]amino}phenyl)-2-phenylpyrimidin-4-yl]phenyl}amino)carbonyl]benzoate (120 mg, 61% yield).

Step 5

To a solution of methyl-3-[({4-[6-(4-{[3-(methoxycarbonyl)benzoyl]amino}phenyl)-2-phenylpyrimidin-4-yl]phenyl}amino)carbonyl]benzoate (180 mg, 0.27 mmol) in THF (3 ml) and MeOH (3 ml) was added 3 ml 5N aqueous KOH solution. The mixture was stirred at room temperature for 5 hrs then and acidified with 20% aqueous HCl solution. The precipitate was washed with water, dried and recrystallized from EtOAc to yield the title compound (145 mg, 84% yield). 1H NMR (DMSO-d6) δ: 7.58 (m, 3H,), 7.67 (m, 2H), 8.04 (d, 4H, J=8.8 Hz), 8.14 (d, 2H, J=8.8Hz), 8.22 (d, 2H, J=7.6 Hz), 8.5 (s, 1H), 8.53 (d, 4H, J=8.8 Hz), 8.55 (s, 2H), 8.68 (m, 2H); ESIMS: m/z 635 (M+H)

### Example 3

Preparation of 2-(3-{6-[3-(5-Carboxy-1,3-Dioxo-1,3-Dihydro-2H-Isoindol-2-Yl)Phenyl]-2-Phenylpyrimidin4-Yl}Phenyl)-1,3-Dioxoisoindoline-5-Carboxylic Acid

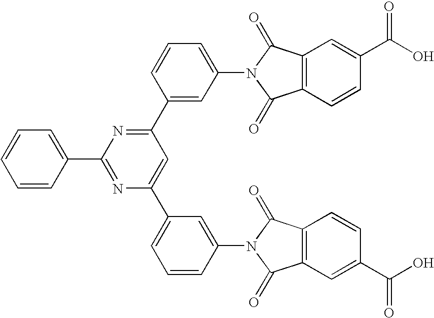

Step1

To a solution of 3-nitroacetophenone (3.3 g, 20 mmol) and 3-nitrobenzaldehyde (3 g, 20 mmol) in acetic acid (50 ml) was added 3.2 ml of concentrated H₂SO₄ at room temperature. The mixture was heated to reflux for 3 hours. The reaction mixture was then cooled to room temperature and quenched with water. The solvent was removed under reduced pressure and the resulting mixture was neutralized with 5N NaOH. The precipitate was filtered, washed with water and then recrystallized from EtOAc/hexanes to give (2E)-1,3-bis(3-nitrophenyl)prop-2-en-1-one (4.8 g).

Step 2

To a solution of (2E)-1,3-bis(3-nitrophenyl)prop-2-en-1-one (1.77 g, 5.94 mmol) and benzamidine hydrochloride (0.5 g, 3.2 mmol) in 95% alcohol (30 ml) was added potassium hydroxide (332 mg, 5.94 mmol) in 95% alcohol (20 ml). The resulting mixture was heated to reflux on a steam-bath for three hours. A solid precipitated from solution after the reaction mixture was cooled to room temperature. The precipitate was thoroughly washed with warm water to remove the inorganic salts followed by warm alcohol. The solid was recrystallized from glacial acetic acid to afford 4,6-bis(3-nitrophenyl)-2-phenylpyrimidine (0.99 g).

Step 3

To a solution of 4,6-bis(3-nitrophenyl)-2-phenylpyrimidine(0.398 g, 1 mmol) in acetic acid (50 ml) was added a catalytic amount of Pd/C under nitrogen. The mixture was bubbled with H₂ gas and stirred for 3 hrs at room temperature. The reaction mixture was filtered through celite. The filtrate was concentrated to provide 3-[6-(3-aminophenyl)-2-phenylpyrimidin-4-yl]aniline (186 mg).

Step 4

A mixture of 100 mg (0.296 mmol) of 3-[6-(3-aminophenyl)-2-phenylpyrimidin4-yl]aniline and 115 mg (0.6 mmol) of 1,2,4 benzenetricarboxylic acid anhydride in 20 ml p-xylene was refluxed for 6 hours. The solvent was removed by heating and the residue was heated at 192° C. for another 3 hours. A yellow solid was formed as the title compound and the yield was quantitative. ¹H NMR (DMSO-d6) δ8.66 (d, d, 2H), 8.60 (s, 2H), 8.58 (s, 1H), 8.56 (d, 2H), 8.43 (d, 2H), 8.33 (s, 2H), 8.11 (d, 2H), 7.78 (t, 2H), 7.69(d, 2H), 7.54 (m, 3H); ESIMS: m/z 687(M+H).

### Example 4

Preparation of 2-(3-{4-[3,4-Bis(2-Methoxyethoxy)Phenyl]-6-[3-(5-Carboxy-1,3-Dioxo-1,3-Dihydro-2H-Isoindol-2-Yl)Phenyl]Pyridin-2-Yl}Phenyl)-1,3-Dioxoisoindoline-5-Carboxylic Acid

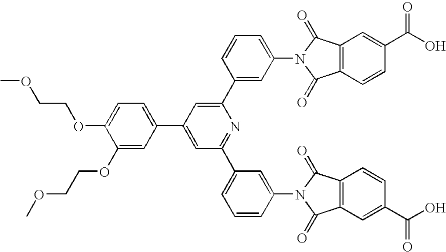

Step 1.

A mixture of 9.9 grams (0.06 mole) of 3-nitroacetonephenone, 4.15 g ( 0.03 mole) of 3,4-dihydroxybenzaldehyde and 30 grams of ammonium acetate in 65 ml glacial acetic acid was refluxed for 4 hours. The reaction mixture was cooled to room temperature and water was added to precipitate the crude product. The precipitate was filtered and washed with acetone, water and hot acetone, and dried to yield 4-[2,6-bis (3-nitrophenyl)pyridin4-yl]benzene-1,2-diol ( 3.68 g) as a yellow solid.

Step 2

A mixture of 1 g. (2.33 mmol) of 4-[2,6-bis(3-nitrophenyl)pyridin-4-yl]benzene-1,2-diol prepared from step 1, 2 grams (14.5 mmol) of potassium carbonate and 800 mg ( 7.36 mmol) of 2-chloroethylmethylether in 30 ml DMF was heated for 10 hrs at 80-100° C. After cooling, the solid was filtered and was washed by DMF. The filtrate was concentrated to dryness in vacuum and then was recrystallized in THF and hexanes to afford 4-[3,4-bis(2-methoxyethoxy)phenyl]-2,6-bis(3-nitrophenyl)pyridine (821 mg) as a yellow solid.

Step 3

To a mixture of 720 mg (1.32 mmol) of 4-[3,4-bis (2-methoxyethoxy)phenyl]-2,6-bis(3-nitrophenyl)pyridine prepared from step 2 and 100mg of palladium on activated carbon (10 wt. %) in 60 ml ethanol was added 2 grams ( 20 mmol) of hydrazine monohydrate under nitrogen atmosphere. The reaction mixture was refluxed for 1 hour. The mixture was filtered and the filtrate was concentrated to dryness which was recrystallized in THF and hexanes to yield 3-{6-(3-aminophenyl)-4-[3,4-bis(2-methoxyethoxy)phenyl]pyridin-2yl}aniline as a white solid (295 mg).

Step 4

A mixture of 49 mg (0.1 mmol) of 3-{6-(3-aminophenyl)-4-[3,4-bis(2-methoxyethoxy)phenyl]pyridin-2yl}aniline, prepared from step 3 and 38.5 mg (0.2 mmol) of 1,2,4 benzentricarboxylic acid anhydride in 5 ml p-xylene was refluxed for 8 hours. After cooling, the resultant solid was filtered and recrystallized in dioxane to afford the title compound as a yellow solid (65 mg). ¹H NMR (DMSO-d6) δ8.41(d, 2H), 8.39 (d, 2H), 8.38 (s, 2H), 8.31 (s, 2H), 8.17 (s, 2H), 8.07 (d, 2H), 7.68 (t, 2H), 7.56(s,1H), 7.55 (d, 2H), 7.54 (d, 1H), 7.12 (d, 2H); ESIMS: m/z 832 (M−H).

The following examples are considered as part of the invention. Such examples illustrate, but are not intended to limit the invention. Such compounds are made using the methods described above. Compounds are arranged in tables by subgroups.

### Example 5

Compounds wherein W is pyridyl, R₁ is R₂ and Ar is a substituted phenyl as described in the table.

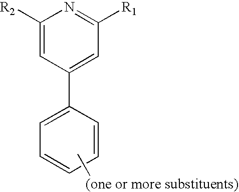

and wherein R₁ and R₂ are

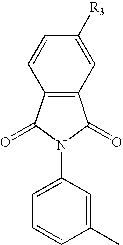

| Ex- | | | |
|---|---|---|---|
| ample 3 | CBM | Phenyl substitution | R₃ |
| A | 000200202 | 3,4,5-trimethoxy | COOH |
| B | 000200275 | 4-(2(diethyl- | COOH |
| | | amino)ethoxy) | |
| C | 000200289 | 3-methoxyethoxy, | COOH |
| | | 4-methoxy | |
| D | 000200297 | 3,4,5-trimethoxy | (trans)-CHCHCOOH |
| E | 000200290 | 3-hydroxy, 4-methoxy | COOH |
| F | 000200280 | 3,5-dimethoxy | COOH |
| G | 000200069 | 4-COOH | COOH |
| H | 000200279 | 4-methoxy | COOH |
| I | 000200068 | (no substitution) | COOH |
| J | 000200049 | 4-methyl | COOH |
| K | 000200276 | 3,4-methylenedioxy | COOH |
| L | 000200291 | 4-methoxyethoxy | COOH |
| M | 000200299 | 3-methoxy | (trans)-CHCHCOOH |
| N | 000200050 | 4-fluoro | COOH |
| O | 000200323 | 4-methoxy, | COOH |
| | | 3-(CH₃O[(CH₂)₂O]₃) | |
| P | 000200277 | 4-hydroxy | COOH |
| Q | 000200278 | 3,4-dihydroxy | COOH |
| R | 000200051 | 4-trifluoromethyl | COOH |
| S | 000200046 | 4-dimethylamino | COOH |
| T | 000200048 | 4-phenyl | COOH |
| U | 000200308 | 3,4,5-trimethoxy | NHC(O)COOH |

### Example 6

The following compounds are also considered to be examples of this invention

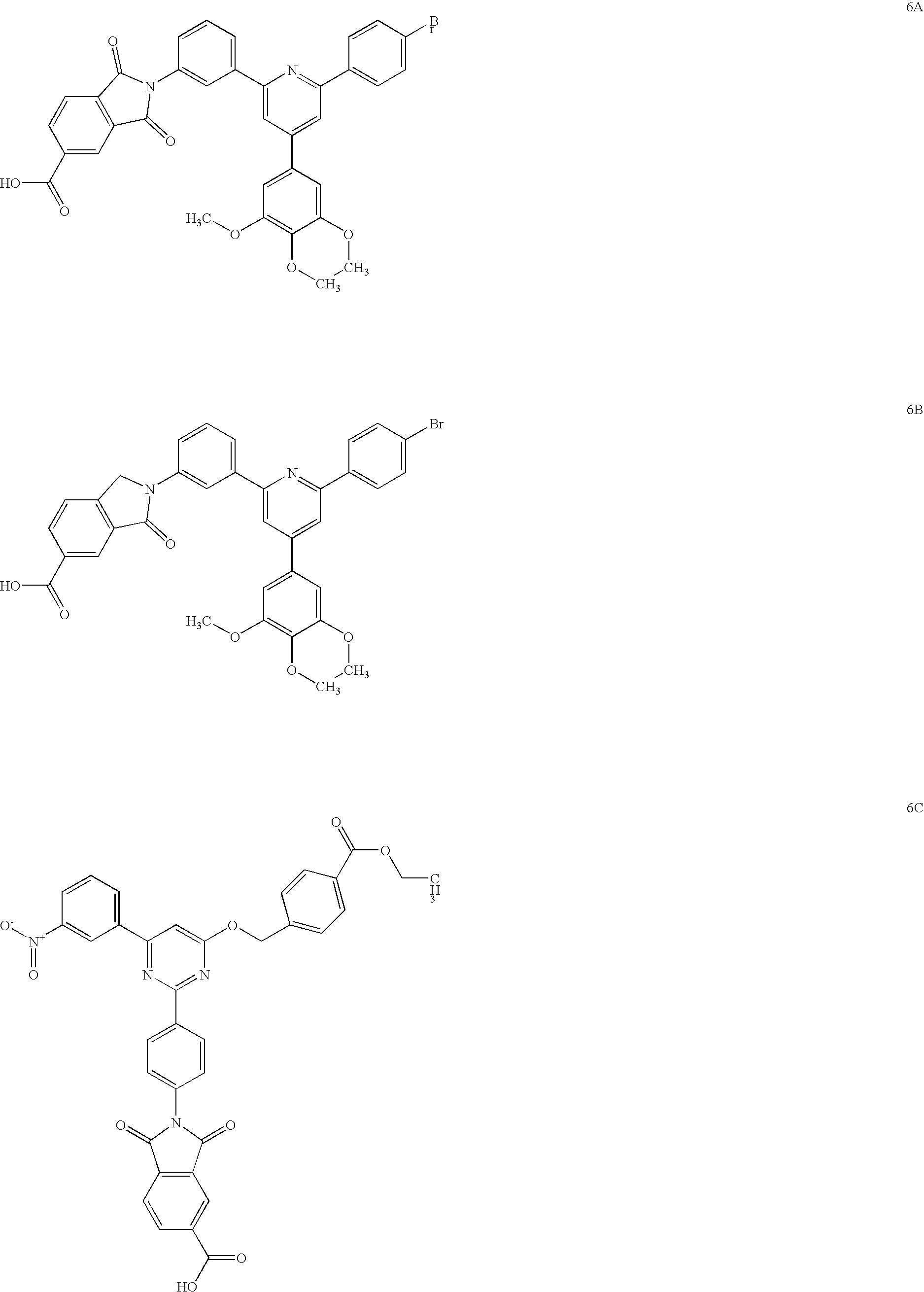

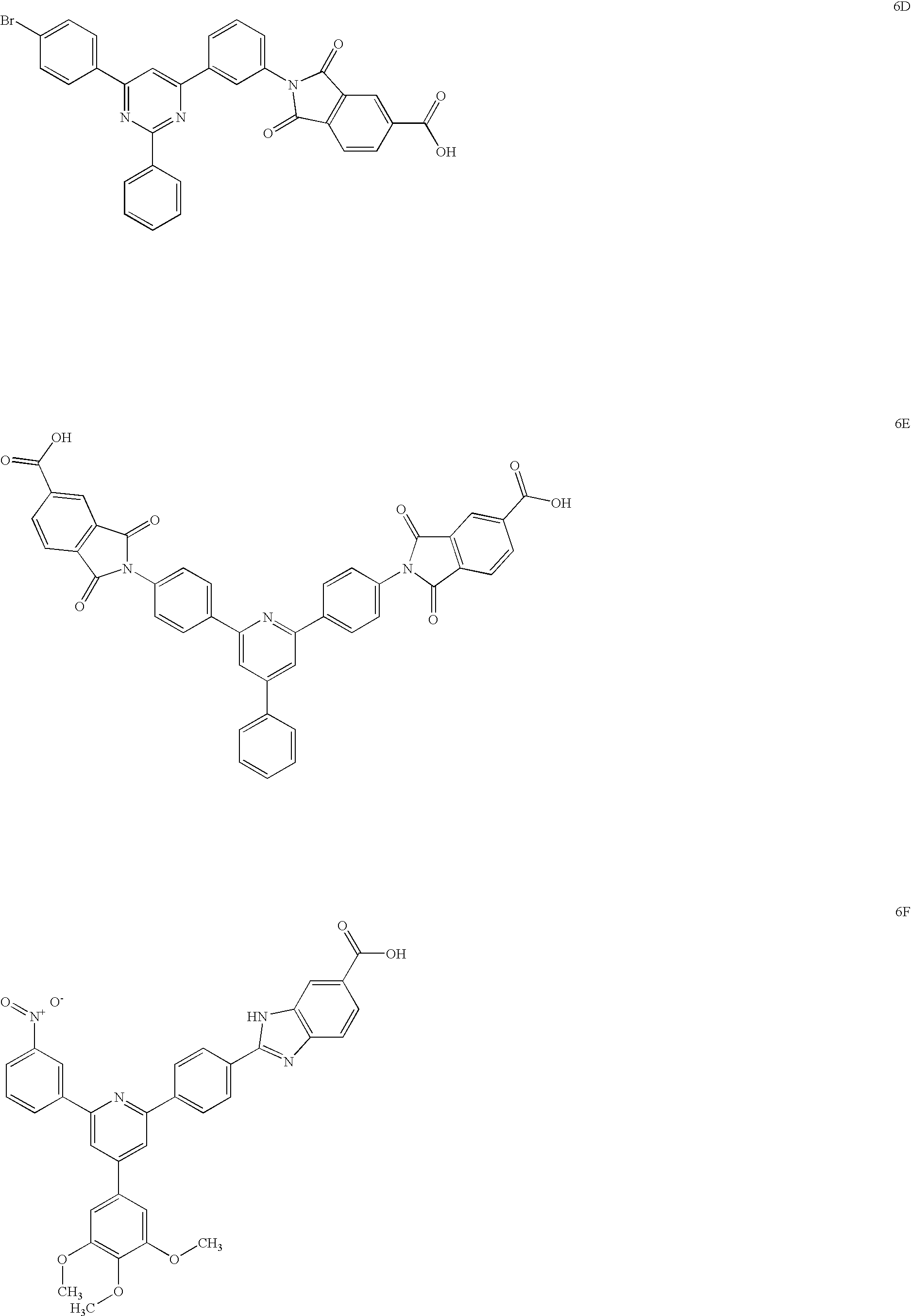

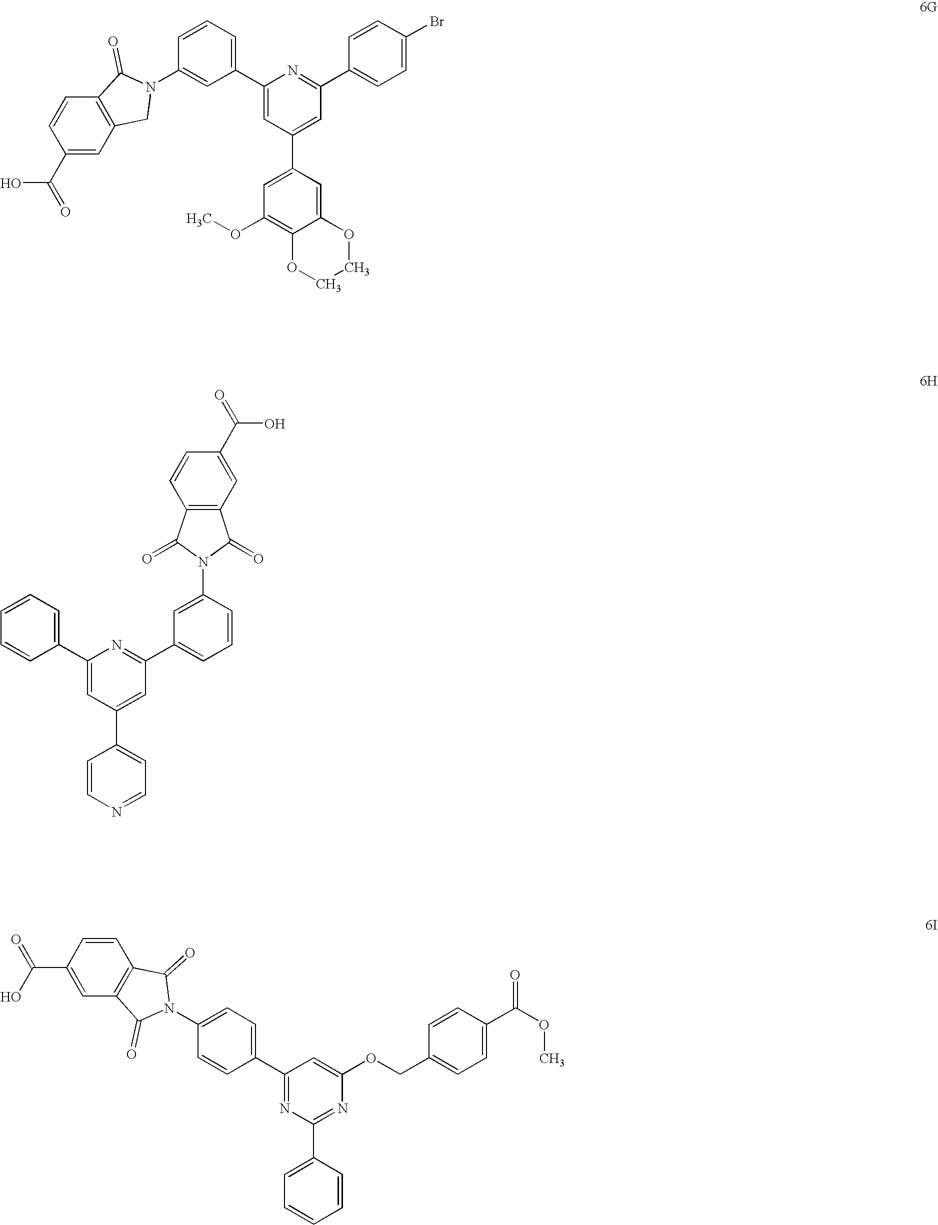

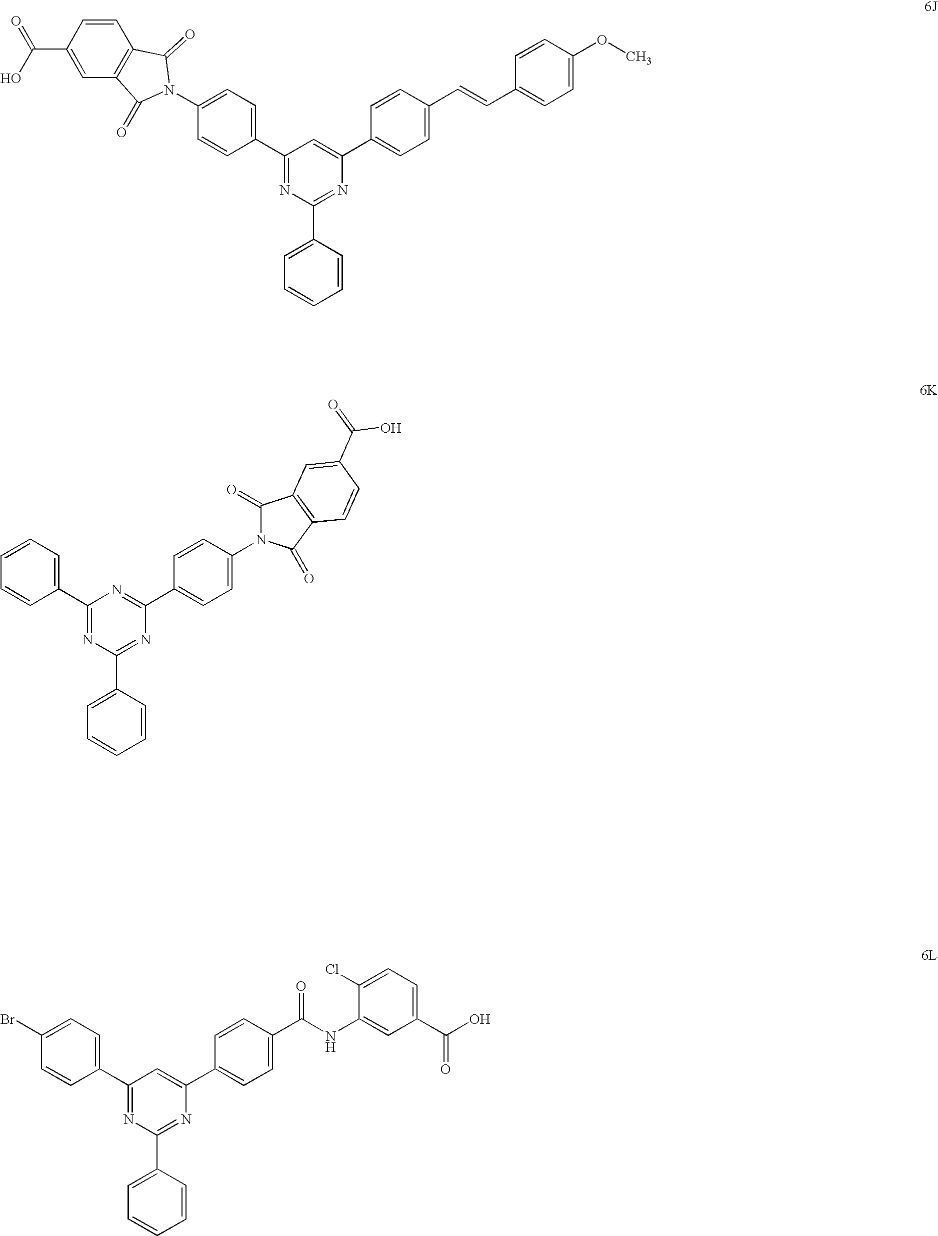

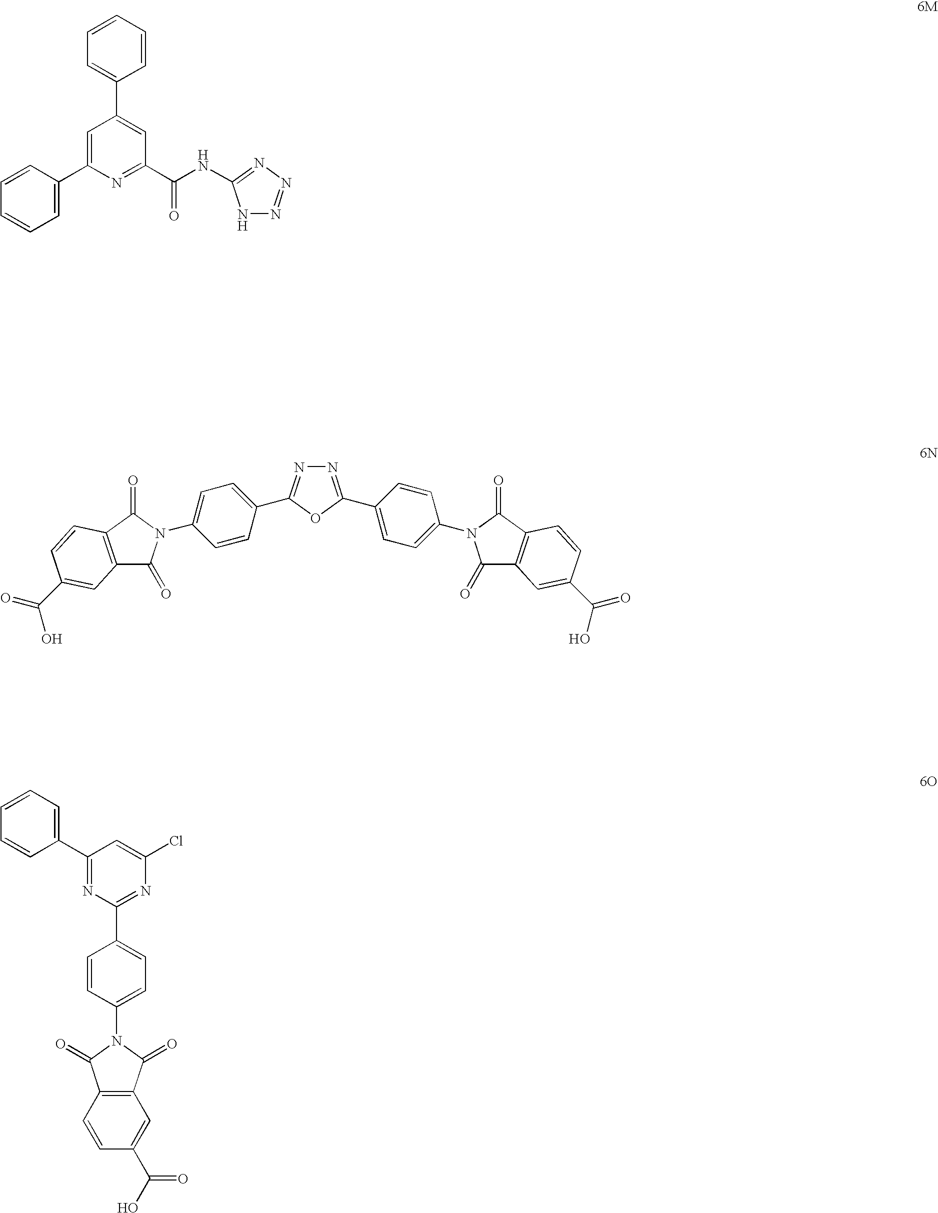

Of course other examples of this invention can be described herein, but the skilled artisan is able to modify the preceding embodiments without undue experimentation, and compositions are within the scope of the skilled artisan in formulation, given the guidance of the specification in light of the state of the art.

All references described herein are hereby incorporated by reference, for example, all patents patent applications, and publications cited are incorporated herein by reference.

Modification of the preceding embodiments is within the scope of the skilled artisan in formulation, given the guidance of the specification in light of the state of the art.

While particular embodiments of this invention have been described, it will be apparent to those skilled in the art that various changes and modifications of this invention can be made without departing from the spirit and scope of the invention. It is intended to cover, in the appended claims, all such modifications that are within the scope of this invention. Hence, the foregoing written specification is considered to be sufficient to enable one skilled in the art to practice the invention. Indeed, various modifications of the above-described makes for carrying out the invention which are obvious to those skilled in the fields of molecular biology, chemistry, medicine, pharmaceutics, or related fields are intended to be within the scope of the following claims.

We claim:

1. The use of a compound of formula: 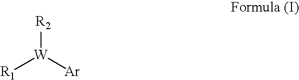
wherein
W is an aromatic core selected from phenyl, pyridyl, pyrimidyl, oxadiazolyl, or triazyl,
Ar is an aromatic substituent, which may be substituted or unsubstituted, where Ar is substituted, it preferably has up to three substituents, and preferably such substituents are in the meta or para position relative to the attachment of Ar to the core molecule, substituents are selected from amino, alkylamino, dialkylamino, loweralkoxy, nitro, carboxy, hydroxy, alkoxy alkyl, alkoxy alkoxy, alkylthio, haloalkyl, halo, lower alkyl, phenyl, pyridyl, an ester, or an amide,
R₁ is selected from, 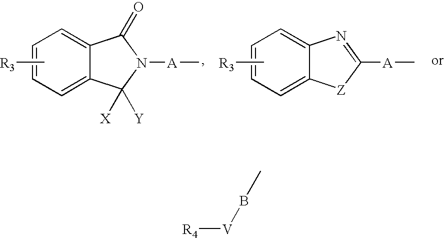
wherein
A is a bond or a spacer selected from phenyl, substituted phenyl, amino, amido, ester, oxy, wherein when phenyl is the spacer, the phenyl spacer is attached to the molecule in the meta or para position,
B is a linker selected from oxy, alkoxy, aryl carbonyl, arylcarbonylamino, a bond, amido, carbonyloxy, oxycarbonyl.
V is an aryl group, selected from phenyl, furyl, thienyl, pyridyl, and pyrrolyl,
X and Y are hydrogen, or together from oxo,
Z is selected from among oxygen, nitrogen, and sulfur,
R₂ is selected from Ar, hydrogen, hydroxy, halo, carboxy or R₁
R₃ is selected from one or two of hydroxy, alkoxy, nitro, sulfoxy, carboxyester, or an amide or such radicals connected to the substituent via a lower alkyl,
R₄ is one or two of amino, alkylamino, dialkylamino, loweralkoxy, nitro, carboxy, hydroxy, alkoxy alkyl, alkoxy alkoxy, alkylthio, haloalkyl, halo, lower alkyl, phenyl, pyridyl, an ester, or an amide, wherein at least one of R₃ and R₄ must be carboxy, or a carboxy substituted radical, and
or pharmaceutically acceptable salt thereof, or a prodrug thereof, or a composition containing such compound, pharmaceutically acceptable salt or prodrug, for the treatment or prophylaxis of diseases by binding to the Flt-1 receptor.

2. The use according to claim 1, of a compound of formula: 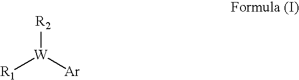
wherein
W is an aromatic core selected from phenyl, pyridyl, pyrimidyl, oxadiazolyl, or triazyl,
Ar is an aromatic substituent, which may be substituted or unsubstituted, where Ar is substituted, it preferably has up to three substituents, and preferably such substituents are in the meta or para position relative to the attachment of Ar to the core molecule, substituents are selected from amino, alkylamino, dialkylamino, loweralkoxy, nitro, carboxy, hydroxy, alkoxy alkyl, alkoxy alkoxy, alkylthio, haloalkyl, halo, lower alkyl, phenyl, pyridyl, an ester, or an amide,
R₁ is selected from, 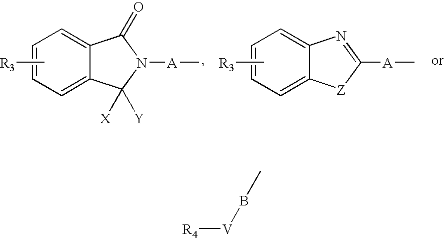
wherein
A is a bond or a spacer selected from phenyl, substituted phenyl, amino, amido, ester, oxy, wherein when phenyl is the spacer, the phenyl spacer is attached to the molecule in the meta or para position,
B is a linker selected from oxy, alkoxy, aryl carbonyl, arylcarbonylamino, a bond, amido, carbonyloxy, oxycarbonyl.
V is an aryl group, selected from phenyl, furyl, thienyl, pyridyl, and pyrrolyl,
X and Y are hydrogen, or together from oxo,
Z is selected from among oxygen, nitrogen, and sulfur,
R₂ is selected from Ar, hydrogen, hydroxy, halo, carboxy or R₁
R₃ is selected from one or two of hydroxy, alkoxy, nitro, sulfoxy, carboxyester, or an amide or such radicals connected to the substituent via a lower alkyl,
R₄ is one or two of amino, alkylamino, dialkylamino, loweralkoxy, nitro, carboxy, hydroxy, alkoxy alkyl, alkoxy alkoxy, alkylthio, haloalkyl, halo, lower alkyl, phenyl, pyridyl, an ester, or an amide, wherein at least one of R₃ and R₄ must be carboxy, or a carboxy substituted radical, and
or pharmaceutically acceptable salt thereof, or a prodrug thereof, or a composition containing such compound, pharmaceutically acceptable salt or prodrug, for the treatment or prophylaxis of diseases treatable by inhibition of angiogenesis.

3. The use according to claim 2, of a compound of formula: 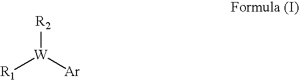
wherein
W is an aromatic core selected from phenyl, pyridyl, pyrimidyl, oxadiazolyl, or triazyl,
Ar is an aromatic substituent, which may be substituted or unsubstituted, where Ar is substituted, it preferably has up to three substituents, and preferably such substituents are in the meta or para position relative to the attachment of Ar to the core molecule, substituents are selected from amino, alkylamino, dialkylamino, lower alkoxy, nitro, carboxy, hydroxy, alkoxy alkyl, alkoxy, alkylthio, haloalkyl, halo, lower alkyl, phenyl, pyridyl, an ester, or an amide,
R₁ is selected from, 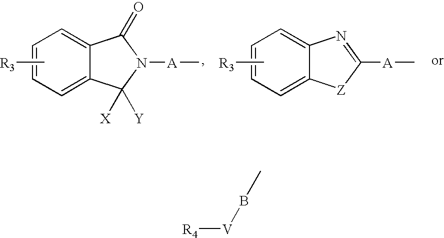
wherein
A is a bond or a spacer selected from phenyl, substituted phenyl, amino, amido, ester, oxy,
B is a linker selected from oxy, alkoxy, aryl carbonyl, arylcarbonylamino, a bond, amido, carbonyloxy, oxycarbonyl.
V is an aryl group, selected from phenyl, furyl, thienyl, pyridyl, and pyrrolyl,
X and Y are hydrogen, or together from oxo,
Z is selected from among oxygen, nitrogen, and sulfur,
R₂ is selected from Ar, hydrogen, hydroxy, halo, carboxy or R₁
R₃ is selected from one or two of hydroxy, alkoxy, nitro, sulfoxy, carboxyester, or an amide or such radicals connected to the substituent via a lower alkyl,
R₄ is one or two of amino, alkylamino, dialkylamino, lower alkoxy, nitro, carboxy, hydroxy, alkoxy alkyl, alkoxy alkoxy, alkylthio, haloalkyl, halo, lower alkyl, phenyl, pyridyl, an ester, or an amide, wherein at least one of R₃ and R₄ must be carboxy, or a carboxy substituted radical, and
or pharmaceutically acceptable salt thereof, or a prodrug thereof, or a composition containing such compound, pharmaceutically acceptable salt or prodrug, for the inhibition of VEGF binding to its receptor Flt-1.

4. The method according to claim 1 wherein:
V is selected from phenyl, furyl, thienyl, pyridyl, and pyrrolyl, and substitution on V is in the 2- or 4- position relative to the point of attachment to the B moiety,
X and Y together are oxo,
R₃ is hydroxy, alkoxy, nitro, sulfoxy, carboxy ester, or an amide or such radicals connected to the substituent via a lower alkyl. In the absence of R3 the ring is substituted only by hydrogen. Preferably only one R₃ appears, and more preferably such R₃ is carboxy.
R₄ is bromo, chloro, and fluoro, fluoroalkyl, methoxy, nitro, carboxy, hydroxy, amino, alkylamino, or —OCH₂O—, wherein at least one of R₃ and R₄ must be carboxy, or a carboxy substituted radical, and
R₂, Ar and R₁ are attached to W via carbons which are not on adjacent atoms in the ring.

5. The method of claim 2 wherein the malady treated is cardiovascular diseases, cancer, diabetic retinopathy, macular degeneration rheumatoid arthritis, skin diseases selected from psoriasis, characterized by hyperplasia and abnormal differentiation of epidermal keratinocytes.

6. A method for the treatment of a patient, comprising administering to the patient the compound in accordance with claim 2 an amount efficacious for inhibition of angiogenesis.

7. A method of claim 2 for the treating cancer, characterized by metastasis in a patient in need of said treatment, comprising administering to the patient the compound an amount efficacious for said treatment.

8. A method of claim 2 for the treatment of cardiovascular diseases in a patient in need of said treatment, comprising administering to the patient the compound an amount efficacious for said treatment.

9. A method for the treatment of a patient, comprising administering to the patient the compound in accordance with claim 3 an amount efficacious for the inhibition of VEGF binding to its receptor Flt-1.

10. The novel pharmaceutical composition used in the method according to claim 3 comprising an amount of a compound efficacious for the inhibition of VEGF binding to its receptor Flt-1, prodrug thereof or pharmaceutical salt thereof; and a pharmaceutically-acceptable carrier.

11. The novel pharmaceutical composition used in the method according to claim 2 comprising an angiogenesis inhibiting compound, prodrug thereof or pharmaceutical salt thereof; and a pharmaceutically-acceptable carrier.

12. The novel pharmaceutical composition of claim 12, comprising a compound of Formula (I).

13. The novel compound of Formula (I) used in the method according to claim 1.

14. The use of a compound according to claim 13 to prepare a medicament.

15. The compound of claim 13 for use as a medicament.

16. A pharmaceutical composition, comprising the compound of claim 15 as an active ingredient.

17. A composition efficacious for the binding to the receptor Flt-1, comprising the compound of claim 13 as an active ingredient.

18. The compound according to claim 13, of formula:

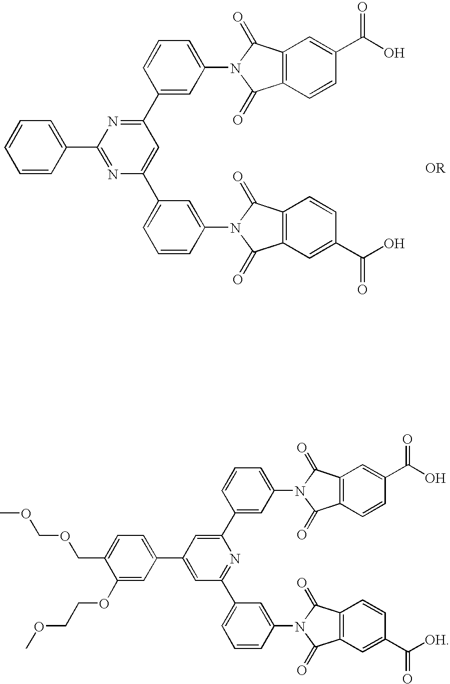

19. The method of claim 1 wherein the compound is of formula: 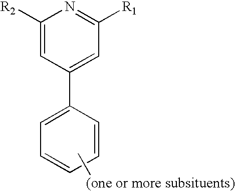
and wherein W is pyridyl, R₁ is R₂ and Ar is a substituted phenyl and wherein R₁ and R₂ are of formula:

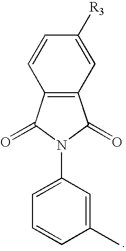

20. The method of claim 1 wherein the binding to the Flt-1 receptor produces a produces an endothelial cell response leading to angiogenesis.

21. The method of claim 1 wherein angiogenesis is promoted.

22. The method of claim 1 wherein the method promotes wound healing, bone healing, collateral circulation, vascularization of skin grafts, healing of burns or ameliorates ischemia.

23. A method for the treatment according to claim 22, comprising administering an amount of a FLT-1 ligand or composition containing a one or more Flt-1 ligands, efficacious for promotion of angiogenesis.

24. A method of stimulating angiogenesis in an animal comprising the method of claim 1.
